(19) United States
(12) Patent Application Publication
(10) Pub. No.: US 2009/0148528 A1
(43) Pub. Date: Jun. 11, 2009

(54) Material that progressively liberates an active liquid substance by evaporation, and mosquito repellent bracelet incorporating such a material

(76) Inventor: Serge Pascal Georges, Noisy Le Sec (FR)

Correspondence Address: BACHMAN & LAPOINTE, P.C., 900 CHAPEL STREET, SUITE 1201, NEW HAVEN, CT, 06510, US

(21) Appl. No.: 11/991,133

(22) PCT Filed: Sep. 4, 2006
(86) PCT No.: PCT/FR2006/002033

§ 371 Date: Feb. 26, 2008

(30) Foreign Application Priority Data
Sep. 2, 2005 (FR) 0508043

(51) Int. Cl. A61K 9/10; A61K 36/00

(52) U.S. Cl. 424/486; 424/725

(57) ABSTRACT

A material controllingly releases by evaporation a liquid active substance and comprises a polymer material matrix impregnated with a liquid composition. The liquid composition contains a mixture of the active substance and a solute which reduces the active substance evaporation kinetics by decreasing a vapor pressure. A method for producing the material and an accessory comprising an element made therefrom are also described.

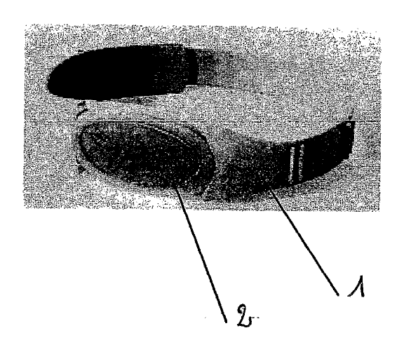

### BACKGROUND

The invention relates to a material that progressively liberates an active liquid substance by evaporation, a manufacturing process for such a material as well as an accessory incorporating an element made of such a material.

The invention applies in particular, without however being restricted to, a bracelet type portable accessory incorporating an element with a repellent function for mosquitoes.

In such an application, the problem that is raised is essentially the life span of the accessory. In fact, even if the active substance is very effective intrinsically, its liberation kinetics are too high to ensure its effectiveness in time.

The invention aims in particular to overcome this problem by proposing a material in which the liberating kinetics of the active substance is slowed down, in a controlled manner by the incorporation of a solution in a quantity that may be adjusted. Consequently, the invention provides an accessory whose life span and effectiveness are optimised.

### SUMMARY OF THE INVENTION

To this end and in a first aspect, the invention proposes a material that progressively liberates an active liquid substance by evaporation, wherein said material comprises a polymer material matrix in which a liquid composition is impregnated. The composition comprises a mixture between the active substance and a solution, wherein said solution is capable of reducing the kinetics of evaporation of the active substance by reducing the vapour pressure.

In a second aspect, the invention proposes a manufacturing process for such a material, in which the impregnation is carried out by a mixture between the matrix and the liquid composition.

In a third aspect, the invention proposes an accessory comprising an element in such a material, wherein said element is an integral part of said accessory.

### BRIEF DESCRIPTION OF THE DRAWINGS

Other specific features and advantages of the invention will become clearer in the following description, made in reference to the attached FIGURE showing in perspective a bracelet according to one embodiment.

### DETAILED DESCRIPTION OF THE PREFERRED EMBODIMENT(S)

In relation to this FIGURE, a portable accessory is described comprising a bracelet 1 onto which an element 2 in the form of a plate is attached. As may be seen in the rest of the description, the plate is made of a material that progressively liberates an active liquid substance by evaporation.

The bracelet 1 is made of a polymer material and incorporates two attachment tabs for the plate which are positioned on either side of it to envelope part of its edges. This creation has the advantage of its manufacturing simplicity, especially by the one piece moulding of the bracelet 1, wherein the plate 2 may then be introduced between the tabs by local deformation of them.

In another embodiment, the plate 2 may be attached by moulding it onto the bracelet 1.

In these creations, the plate 2 is positioned on the bracelet 1, there is no direct contact between the material of the plate 2 and the skin of the person wearing said bracelet, so as to avoid in particular any cutaneous allergic reaction.

Even though the description is made in relationship to this specific bracelet 1, the invention is in no way restricted to this configuration, nor to a bracelet, nor to an accessory designed to be worn against the skin. In fact, any type of portable accessory may be envisaged, whether this is to be worn by a human or by an animal. It is also possible to make accessories that are not specifically designed to be worn, for example accessories that may be placed or attached to furniture or hung up. In particular, the accessory may comprise in general a bearing structure on which an element made of a liberating material is attached to become an integral part of said accessory.

The liberating material comprises a matrix in polymer material in which a liquid composition is impregnated. The composition comprises a mixture between the active substance to be liberated and a solution. The solution is capable of reducing the kinetics of evaporation of the active substance by reducing its vapour pressure.

In one embodiment, the matrix comprises a copolymer ethylene vinyl acetate (EVA) resin, especially comprising a large quantity of vinyl acetate grouping. For example, the resin comprises between 18% and 42% in weight of vinyl acetate grouping. The use of a resin sold by the ARKEMA company under the name EVATANE® provides good results as concerns the liberation kinetics of the active substance.

The polymer matrix may comprise, possibly in addition to the resin mentioned above, a polypropylene (PP) based resin. In the case of joint use, a weight ratio of around 2 EVA for 1 PP provides good results within the scope of the invention.

In one example of an embodiment, the active substance has a repellent function for pests, in particular for mosquitoes.

To achieve this, the active substance may comprise an essential or a mixture of essential oils, especially chosen in the group comprising citronella essential oil, hybrid lavender essential oil, geranium essential oil, turpentine essential oil, clove essential oil, peppermint essential oil and patchouli essential oil. In one embodiment, the active substance comprises a mixture of all the essential oils mentioned above, in weight proportions which decrease in the order they are mentioned.

Tests have been carried out and proven the effectiveness especially of the use of a solution comprising at least one food substance to reduce the kinetics of evaporation of the active substance.

In particular, the food substance is selected from menthol carboxamide, the vanillin or a mixture of these two compounds. In the case of the mixture being used, a weight ratio between the menthol carboxamide and the vanillin of between 4/1 and 2/1 provides particularly good results.

The material according to the invention may comprise between 20% and 80% in weight of impregnated liquid composition, wherein said composition may comprise between 10% and 30% in weight of solution. However, matrices may be impregnated with more than 100% in weight of liquid composition, and compositions comprising a different quantity of solution may be made to suit the specific application in question for the material.

Furthermore, the material may comprise known additives, such as colouring or formatting agents.

According to the invention, it is possible to conserve the effectiveness of the impregnated material for one to two months, once the packaging has been opened. That is to say that the evaporation kinetics permit the progressive liberation of the active substance during this entire period. Furthermore, by adjusting the formulation of the composition and/or the matrix, it is possible to adjust the duration of the liberation of the active substance to suit the specific application in question.

As concerns the manufacturing process of the material described above, the impregnation is carried out by mixing the matrix and the liquid composition. The mixture may be made over quite a long period, for example several days, in order to ensure optimal impregnation. Furthermore, the mixture may be carried out at ambient temperature in a sealed chamber or in a vacuum in order to improve the conservation of the effectiveness of the composition.

Prior to mixing, the liquid composition is formed by dissolving the solution in the active substance. For example, the solution may be in the solid form, especially a powder, and the active substance may be heated moderately and agitated to improve its dissolution in the solution.

After mixing, the impregnated material may be formatted, for example in the form of a plate as shown in the FIGURE, according to known techniques such as injection or extrusion without any particular constraints.

In this way, the material element may be in any form desired for better integration into the accessory that is to comprise it. As a variant, the accessory may be entirely made from impregnated material as it has the mechanical cohesion provided by the matrix. It may also be envisaged to mould on the impregnated material with a compatible material so as to form an accessory integrating this moulded on part.

1-14. (canceled)

15. A material that progressively liberates an active liquid substance by evaporation, wherein said material comprises a matrix of polymer material in which a liquid composition is impregnated the liquid composition comprising a mixture of an active substance and a solution, and said solution being capable of reducing evaporation kinetics of the active substance by reducing vapor pressure.

16. The material according to claim 15, wherein the matrix comprises a ethylene-vinyl acetate copolymer resin (EVA).

17. The material according to claim 16, wherein the resin comprises between 18% and 42% in weight of a vinyl acetate grouping.

18. The material according to claim 15, wherein the matrix comprises a polypropylene based resin.

19. The material according to claim 15, wherein the active substance has a repellent function for pests.

20. The material according to claim 19, wherein the active substance comprises at least one essential oil selected from the group consisting of citronella essential oil, geranium essential oil, hybrid lavender essential oil, turpentine essential oil, clove essential oil, peppermint essential oil and patchouli essential oil.

21. The material according to claim 15, wherein the solution comprises at least one food substance selected from the group consisting of menthol carboxamide, vanillin and a mixture thereof.

22. The material according to claim 15, wherein the material comprises between 20% and 80% in weight of the impregnated liquid composition.

23. The material according to claim 15, wherein the liquid composition comprises between 10% and 30% in weight of solution.

24. A manufacturing process for forming a material that progressively liberates an active liquid substance by evaporation, wherein said material comprises a matrix of polymer material in which a liquid composition is impregnated the liquid composition comprising a mixture of an active substance and a solution, and said solution being capable of reducing evaporation kinetics of the active substance by reducing vapor pressure comprising carrying out impregnation of the liquid composition by mixing the matrix and the liquid composition.

25. The manufacturing process according to claim 24, further comprising prior to impregnation, forming the liquid composition by dissolution of the solution in the active substance.

26. An accessory comprising an element containing a material that progressively liberates an active liquid substance by evaporation, wherein said material comprises a matrix of polymer material in which a liquid composition is impregnated the liquid composition comprising a mixture of an active substance and a solution, and said solution being capable of reducing evaporation kinetics of the active substance by reducing vapor pressure wherein said element is an integral part of said accessory.

27. The accessory according to claim 26, further comprising a support structure onto which the element is attached.

28. The accessory according to claim 26, further comprising a bracelet onto which the element in the form of a plate is attached, and the material contained in said element having a repellent function for mosquitoes.
